(19) United States
(12) Patent Application Publication
(10) Pub. No.: US 2010/0260887 A1
(43) Pub. Date: Oct. 14, 2010

(54) Content of the essential amino acids lysine and methionine in algae and cyanobacteria for improved animal feed

(75) Inventors: Shai Ufaz, Givat-Ada (IL); Jonathan Gressel, Rehovot (IL)

Correspondence Address: DODDS & ASSOCIATES, 1707 N STREET NW, WASHINGTON, DC, 20036, US

(73) Assignee: TransAlgae, Rehovot (IL)

(21) Appl. No.: 12/658,815

(22) Filed: Feb. 16, 2010

Related U.S. Application Data
(60) Provisional application No. 61/207,825, filed on Feb. 17, 2009.

(51) Int. Cl. A23K 1/00; C12N 1/21; C12N 1/13

(52) U.S. Cl. 426/7; 435/252.3; 435/257.2; 426/61

(57) ABSTRACT

This disclosure provides a method to improve lysine and methionine content of algae and cyanobacteria through genetic modification in combination with modified expression of high lysine and methionine proteins as sinks for the amino acids. The method of this disclosure is specifically useful in animal feed production.

### PRIORITY

This application claims priority of U.S. provisional application No. 61/207,825 filed on Feb. 17, 2009 and of U.S. nonprovisional application Ser. No. 12/584,571 filed on Sep. 8, 2009.

### SEQUENCE LISTING

This application contains sequence data provided on a computer readable diskette and as a paper version. The paper version of the sequence data is identical to the data provided on the diskette.

### FIELD OF THE INVENTION

This invention relates to the field of genetically engineering algae and cyanobacteria. More specifically the invention relates to improving the amino acid content of algae and cyanobacetria for use as animal feed.

### BACKGROUND OF THE INVENTION

Most present protein sources for mono-gastric animals (including those cultivated in aquaculture) are specifically deficient in components necessary for a balanced diet. A balanced amino-acid composition for fish, mammals, and fowl is typically obtained by mixing various grains and fishmeal, each to overcome the deficiencies of the others, and/or by adding synthetic amino acids. This seems not to be effective for aquaculture, where large proportions of fishmeal must be added to the diet. As the aquaculture industry is rapidly growing in the last several years, fishmeal and fish oil supplies are insufficient and dwindling affecting the future growth of aquaculture production, especially of carnivorous fish. The essential amino acids lysine and methionine are the major limiting factors in substitutes for fishmeal such as soybeans. Soybeans can be used as only a small part of fish diets, possibly because soybean also contains antifeedants. Whereas synthetic DL methionine can be added to the diet of terrestrial mono-gastric animals, its soluble nature precludes its use in pellets for penned fish, unless complexed with calcium to achieve a poorly soluble salt.

Initial diets for aquaculture species typically contain high levels of fishmeal and fish oil, which are required ingredients for carnivorous fish and other seafood species. Additionally, fishmeal is a high protein ingredient with a good quality balance of essential amino acids and fish oil also contains n-3 (omega 3) fatty acids, required by many aquatic animals. The aquatic medium does not contain a high percentage of carbohydrates available as calories, so carbohydrate content is of lesser importance. Given this generalization, it is not surprising that most aquatic animals grow best when fed relatively high levels of crude protein and lipid, and that balanced essential amino acid and fatty acid concentrations in the diet are high priority considerations when formulating diets. However, the dwindling fishmeal and fish oil supplies are insufficient to realize growth in aquaculture production, and finding even partial replacements that are better than soybean meal are imperative.

The inability of humans and other monogastric species to synthesize certain amino acids has long triggered tremendous interest in increasing the levels of these essential amino acids in crop plants. Knowledge obtained from basic genetics and genetic engineering research has also been successfully used to enrich the content of some of these essential amino acids in crop plants, but this often renders them more susceptible to pathogen, insect, and rodent attack. The progenitors of crops typically have grain with more balanced amino acid contents; there was a selective value in pest resistance to lose at least one amino acid during domestication (Morris and Sands, 2006). Among the essential amino acids, lysine (Lys), tryptophan (Trp), and methionine (Met) have received the most attention because they are most limiting in cereal and leguminous crops, which represent the major vegetarian sources of human food and animal feed worldwide.

One way to complement the essential amino acid profile of a crop is to express natural proteins from different species that contain sufficient quantities of the desired essential amino acids (heterologous expression). Simple expression of a methionine-rich maize protein in a methionine-deficient legume or of a lysine-rich legume protein in lysine-deficient soybean would generate a seed that could function as a more complete protein source, if possible. But as noted above, their cultivation in practice is problematic. A number of proteins have been identified as methionine-rich sources: the maize 10-kDa zein with 30% methionine (Kirihara et al., 2001; and references cited therein); the maize 15-kDa zein with 15% methionine (Pedersen et al., 1986); 2S albumin from Bertholletia exalsa (Brazil nut) harboring 24% methionine and a 10-kDa seed prolamin with 25% methionine by weight (Masumura et al., 1989); and an 18-kDa zein (high-sulfur zein) with 37% methionine (Chui et al., 2003).

### SUMMARY OF THE INVENTION

The current invention provides a solution to the above described flaws of the present day technologies.

Algae and cyanobacteria have the potential to supply the growing needs for fishmeal either directly or as feed for zooplankton. Improving the content of the essential amino acids lysine and methionine in algae and cyanobacteria using genetic engineering techniques will significantly improve the nutritional quality of alga/cyanobacteria as partial or maybe even complete fishmeal replacements and can be of even greater nutritional value than fishmeal itself, as their oil composition is also similar to that of fish oil. This could become the solution for the high demand for aquaculture production of high value carnivorous fish and other seafood species over the next decades, as well as a replacement of soybean in animal and poultry diets. Intensively, axenically cultivated algae and cyanobacteria do not have the problems of pest attack that is so problematic in agricultural field crops.

Accordingly, this invention provides a method to increase essential amino acids in algae and cyanobacteria for producing nutritionally rich proteins for fish food and animal feed. The genetically modified algae could serve as direct source food for fish or do so indirectly through zooplankton. This is achieved by together in combination modifying the biosynthesis pathway of lysine and methionine together with expression of high methionine and lysine proteins modified for expression in algae/cyanobacteria and serve as sink for these essential amino acids. These modifications will also be applied to algae/cyanobacteria with reduced level of Rubisco (ribulose 1-5 bis phosphate carboxylase/oxygenase), which has a relatively low level of these essential amino acids but constitutes major part of the cell protein.

According to one preferred embodiment of the invention, a transgenic alga or cyanobacterium expressing recombinant protein with high methionine and/or lysine content is generated by genetic engineering.

According to one preferred embodiment the transformation of the alga or cyanobacterium is achieved by microporation.

According to another preferred embodiment an animal feedstock is produced by transforming cyanobacteria or algae with polynucleotide sequences encoding for high lysine and/or methionine proteins.

According to yet another preferred embodiment recombinant proteins are used as animal feed

### A SHORT DESCRIPTION OF THE DRAWINGS

FIG. 1: The D-AtCGS coding sequence fused to Chlamydomonas rbcS chloroplast transit peptide and 3xHA epitope tag, chemically synthesized according to Chlamydomonas codon usage and cloned downstream to the Chlamydomonas HSP70-rbcS promoter and upstream to the rbcS terminator. The 3xHA tag is used for detection of the protein in the absence of antibodies. It is designed in a way that will enable the removal of the tag and transform the construct with and without the HA tag.

FIGS. 2 A and B: The Zea mays delta zein 15 kD gene fused to 3xHA tag (A) or fused to Zea mays delta zein 10 kD using the hinge region of anti HSV antibody (accession number: AY191459) as a linker (B), cloned downstream to Chlamydomonas HSP70-rbcS promoter and rbcS terminator.

FIG. 3: The Zea mays delta zein 15 kD coding sequence chemically synthesized according to Chlamydomonas chloroplast codon usage fused to 3xHA tag and cloned under the control of the chloroplast atpA promoter and rbcL terminator.

FIG. 4: Corynebacterium dapA gene fused to rbc TP and 3xHA epitope tag cloned downstream to Chlamydomonas HSP70-rbcS promoter and 35S terminator.

FIG. 5: The barley high lysine 8 protein (BHL8) de novo synthesized according to Chlamydomonas codon usage and cloned under Chlamydomonas HSP70-rbcS promoter in plasmid containing the phytoene desaturase (pds) gene conferring resistance to phytoene desaturase inhibiting herbicide.

FIG. 6: The Zea mays delta zein 15 kD gene fused to 3xHA tag cloned downstream to P. tricornutum fcpB promoter and upstream to fcpB terminator in the plasmid pPhaT (Falciatore et al., 1999).

FIG. 7. Western blot analysis of Chlamydomonas colonies transformed with the plasmid pSI-Zein Fusion containing the zein fusion cassette under the control of the Chlamydomonas HSP70-rbcS fusion promoter. A band corresponding to a protein of ˜40 kD that is detected by the specific anti-HA antibody is circled.

### DETAILED DESCRIPTION OF THE INVENTION

Algae and cyanobacteria with biotechnological utility are chosen from among the following, non-exclusive list of organisms:

Pavlova lutheri, Isochrysis CS-177, Nannochloropsis oculata CS-179, Nannochloropsis like CS-246, Nannochloropsis salina CS-190, Nannochloropsis gaditana, Tetraselmis, Tetraselmis suecica, Tetraselmis chuii and Nannochloris spp., Chlamydomonas reinhardtii as representatives of all algae species. The phylogeny of the algae is summarized in Table 1. Synechococcus PCC7002, Synechococcus WH-7803, Thermosynechococcus elongaues BP-1 are used as representatives of all cyanobacterial species.

TABLE 1
Phylogeny of some of the algae used

| Genus | Family | Order | Phylum | Sub-Kingdom |
|---|---|---|---|---|
| Chlamydomonas | Chlamydomonadaceae | Volvocales | Chlorophyta | Viridaeplantae |
| Nannochloris | Coccomyxaceae | Chlorococcales | Chlorophyta | Viridaeplantae |
| Tetraselmis | Chlorodendraceae | Chlorodendrales | Chlorophyta | Viridaeplantae |
| Phaeodactylum | Phaeodactylaceae | Naviculales | Bacillariophyta | Chromobiota |
| Nannochloropsis | Monodopsidaceae | Eustigmatales | Heterokontophyta | Chromobiota |
| Pavlova | Pavlovaceae | Pavlovales | Haptophyta | Chromobiota |
| Isochrysis | Isochrysidaceae | Isochrysidales | Haptophyta | Chromobiota |

Phylogeny according to: http://www.algaebase.org/browse/taxonomy/
Note:
Many genes that in higher plants and Chlorophyta are encoded in the nucleus are encoded on the chloroplast genome (plastome) of Chromobiota, red lineage algae (Grzebyk, et al. (2003).

Attaining Algae/Cyanobacteria with High Lysine:

The coding region of feedback insensitive bacterial DHDPS (Corynebacterium dihydrodipicolinate synthase) (SEQ ID NO: 1) is expressed together with RNAi of algal LKR/SDH (lysine-ketoglutarate reductase/saccharopine dehydrogenase) (SEQ ID NO: 2, or LKR/SDH from any other algae) as described previously (Zhu and Galili, 2004), together with genetically engineered gene encoding a protein with high lysine designed according to the codon usage of the algae/cyanobacyeria such as BARLEY HIGH LYSINE8 (BHL8) protein (Jung and Carl, 2000) (SEQ ID NO: 3) (U.S. Pat. No. 7,211,431, but different codon usage) or synthetic coiled-coil high-lysine/high-methionine proteins (SEQ ID NO:4) (Keeler et al., 1997) (U.S. Pat. No. 5,773,691) or the Amaranthus hypochondriacus AmA1 seed protein (Accession no: AF49129, Chakraborty et al., 2000) (SEQ ID NO: 5) (U.S. Pat. No. 5,846,736).

These proteins are known to accumulate in transgenic potato tubers or maize or tobacco seeds. BHL8 is a recombinant protein derived from a barley CHYMOTRYPSIN INHIBITOR-2, which was genetically engineered to substantially increase the number of Lys codons and those of other essential amino acids, based on a three-dimensional structure analyses (Roesler and Rao, 2000).

Attaining Algae/Cyanobacteria with High Methionine:

Again the strategy is to enhance the ability to synthesize methionine together with the expression of a methionine-rich recombinant protein designed to be expressed in algae/cyanobacteria, according to specific codon usage of each. A high level of free methionine is achieved by overexpression of a mutated form of Arabidopsis cystathionine γ-synthase (D-AtCGS) (SEQ ID NO: 6) (U.S. patent application Ser. No. 10/475,852, but different codon usage, different chloroplast transit peptide) the enzyme that controls the synthesis of the first intermediate metabolite in the methionine pathway (Hacham, 2008 and U.S. patent application Ser. No. 10/475,852). The mutated AtCGS is expressed together with high methionine protein, such as 2S albumin from Amaranthus hypochondriacus (SEQ ID NO: 5) or the HetR gene encoding protein from Anabaena sp. strain PCC 7120 (SEQ ID NO: 7) or the delta zein structural 15 protein, accession NO: AF371264 (SEQ ID NO: 8). The DNAs encoding these proteins with modified codon usage are transferred to algae/cyanobacteria nuclear and/or chloroplast genomes for expression under a strong constitutive or inducible promoters such as RbcL, RbcS, 35S, ubiquitin, nitrate reductase, HSP70 for nuclear transformation and rbcL, psaD, psaB, and atpA promoters for chloroplast transformation.

Attaining Algae/Cyanobacteria with High Lysine and Methionine in Proteins:

A gene encoding a synthetic protein with high methionine and high lysine is de novo synthesized and transformed into algae/cyanobacteria alone or together with truncated Arabidopsis cystathionine γ-synthase (D-AtCGS) (SEQ ID NO: 6) or feedback insensitive bacterial DHDPS (dihydrodipicolinate synthase) (SEQ ID NO: 1). Such a protein encoding gene can be the high methionine Anabaena HetR gene linked to a high lysine α-helical coiled coil protein, as described in the examples below.

Additional strategy for obtaining transgenic algae or cyanobacteria rich in these essential amino acids is to express the proteins mentioned above in transgenic algae or cyanobacteria modified to express reduced amount of Rubisco protein. Rubisco protein has relatively low content of essential amino acids, including methionine and lysine, but it constitutes major part of the cell proteome. Accordingly, reduced Rubisco content in the cell would allow ‘space’ for recombinant high lysine and/or methionine containing proteins. Details of transgenic algae expressing low levels of Rubisco are disclosed in U.S. provisional patent application U.S. 61/191,453 and non-provisional application U.S. Ser. No. 12/584,571 that are both incorporated herein by reference.

The methodology used in the various steps of enabling the invention is described below:

### Nucleic Acid Extraction

Genomic DNA is isolated using either Stratagene's (La Jolla, Calif.) DNA purification kit or a combination of QIAGEN's (Valencia, Calif.) DNeasy plant mini kit and phenol chloroform extraction (Davies et al. 1992). Total RNA is isolated using either QIAGENS's Plant RNeasy Kit or the Trizol Reagent (Invitrogen, Carlsbad, Calif.).

### Transformation of Algae

Chlamydomonas CW15 wild type or the arginine deficient mutant (CC-425) were transformed with the plasmid from examples 1 and 2 (1±5 mg) by the glass bead vortexing method (Kindle, 1990). The transformation mixture was transferred to 50 mL of non-selective growth medium for recovery and incubated for at least 18 h at 25° C. in the light. Cells were collected by centrifugation and plated at a density of 10⁸ cells per Petri dish. Transformants were grown on fresh TAP or SGII agar plates containing a selection agent for 7-10 days in 25° C.

The diatom Phaeodactylum tricornutum was transformed by microparticle bombardment using a Biolistic PDS-1000/He Particle Delivery System (Bio-Rad, Hercules, Calif., USA) as previously described (Falciatore et al., 1999). For selection of transformant, bombarded cells were plated onto 50% artificial sea water (ASW)+f/2 agar plates (1% agar) supplemented with 100 μg/ml phleomycin (InvivoGen, San Diego, Calif., USA). After about three weeks of incubation under white light, 22-25° C., individual resistant colonies were restraked on 100% ASW+f/2 agar plates, supplemented with 100 μg/ml zeocin (Invitrogen, Carlsbad, Calif., USA) and inoculated into liquid ASW+f/2 medium to be further analyzed.

Other marine algae are transformed using microporator as described below: A fresh algal culture is grown to mid exponential phase in ASW+f/2 media. A 10 mL sample of the culture is harvested, washed twice with Dulbecco's phosphate buffered saline (DPBS, Gibco) and resuspended in 250 μl of buffer R (supplied by Digital Bio, Seoul, Korea, the producer of the microporation apparatus and kit). After adding 8 μg linear DNA to every 100 μl cells, the cells are pulsed. A variety of pulses is usually needed, depending on the type of cells, ranging from 700 to 1700 volts, 10-40 ms pulse length; each sample is pulsed 1-5 times. Immediately after pulsing the cells are transferred to 200 μl fresh growth media (without selection). After incubating for 24 hours in low light, 25° C., the cells are plated onto selective solid media and incubated under normal growth conditions until single colonies appear.

### Transformation of Cyanobacteria

For transformation to Synechococcus PCC7002, cells are cultured in 100 mL of BG-11+

Turks Island Salts liquid medium (http://www.crbip.pasteur.fr/fiches/fichemedium.jsp?id=548) at 28° C. under white fluorescent light and cultured to mid exponential growth phase. To 1.0 mL of cell suspension containing 2×10⁸ cells, 0.5-1.0 μg of donor DNA (in 10 mM Tris/1 mM EDTA, pH 8.0) is added, and the mixture is incubated in the dark at 26° C. overnight. After incubation for a further 6 h in the light, the transformants are selected on BG-11+Turks Island Salts 1.5% agar plates containing a selection agent until single colonies appear.

There is no prior art known to us of previously transforming the following species, except by the research group of the inventors of this application: Pavlova lutheri, Isochrysis CS-177, Nannochloropsis oculata CS-179, Nannochloropsis like CS-246, Nannochloropsis sauna CS-190, Tetraselmis suecica, Tetraselmis chuii and Nannochloris sp. nor has microporation been used previously for transforming algae cyanobacteria or higher plants.

### Protein Extraction

1 to 10 mL cells at 5×10⁶ cell/mL are harvested and resuspended in 500 μl extraction buffer (50 mM Tris pH=7.0; 1 mM EDTA; 100 mM NaCl; 0.5% NP-40; and protease inhibitor (Sigma cat# P9599). Then 100 μl of glass beads (425-600 μm, Sigma) are added and cells are broken in a bead beater (MP FastPrep-24, MP Biomedicals, Solon, Ohio, USA) for 20 sec. The tube content is centrifuged for 15 min, 13000×g, at 4° C. The supernatant is removed to new vial for quantification and western blot analysis.

For extraction of the zein protein, the soluble part of the extract is removed and the pellet is resuspended with 70% ethanol and 1% β-mercaptoethanol. The zein fraction is then extracted by incubation in 65° C. for 30 min and the tube is centrifuged for 30 min in 4° C., 13000×g. The ethanol is then evaporated from the sample with nitrogen gas and loading buffer is added before loading the gel.

### Protein Separation by PAGE and Western Analysis

Extracted proteins are separated on a 4-20% gradient SDS-PAGE (Gene Bio-Application Ltd., Kfar Hanagid, Israel), at 160V for 1 hr. They were then either stained by Coomassie (Sigma) or blotted onto PVDF (Millipore, Billerica, Mass., USA) membranes for 1 h at 100 volts in the transfer buffer (25 mM Tris, 192 mM glycine and 20% methanol). The proteins are detected with the anti HA antibody (Sigma catalog no. H9658) diluted to a ratio of 1:1000 in antibody incubation buffer (5% skim milk, Difco). An alkaline phosphatase conjugated anti-rabbit antibody (Millipore, Billerica, Mass., USA), at 1:10000 dilution in the same buffer was used as a secondary antibody. Detection was carried out using the standard alkaline phosphatase detection procedure (Blake et al., 1984).

### Amino Acid Analyses

The amino acid composition wild type and transgenic algae/cyanobacteria is determined by using a C18 HPLC column equipped with an online Pico Tag amino acid analyzer (Waters). Total soluble protein from wild-type and transgenic algae is precipitated with 10% trichloroacetic acid on ice for 45 min, washed with ethanol-diethylether (1:1, vol/vol), and lyophilized. Acid hydrolysis and derivatization of lyophilized protein with phenyl isothiocyanate (PITC) is performed as per the Pico Tag manual. The PITC derivative of each amino acid was detected by absorbance at 254 nm.

The invention is now described by means of various non-limiting examples:

### Example 1

### Expression of Mutated Form of Cystathionine γ-Synthase (CGS) (SEQ ID NO: 15), Together with Zea mays Delta Zein 15 Kd Protein Alone (SEQ ID NO: 16) or Fused to Zea mays Delta Zein 10 kD Protein (SEQ ID NO: 17)

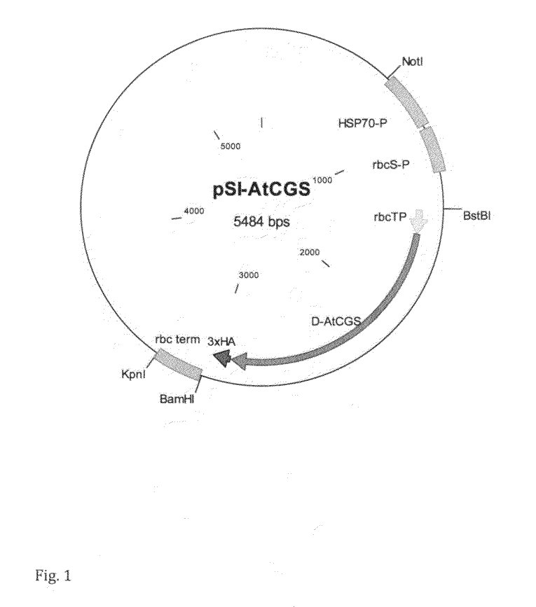

The D-AtCGS coding sequence (Hacham et al., 2006), fused to Chlamydomonas rbcS chloroplast transit peptide (SEQ ID NO: 13) and 3xHA epitope tag, was chemically synthesized according to Chlamydomonas codon usage (SEQ ID NO: 14) and cloned downstream to the Chlamydomonas HSP70-rbcS fusion promoter and upstream to rbcS terminator in the plasmid pSI103 (Sizova et al., 2001) replacing the aphVIII gene (FIG. 1).

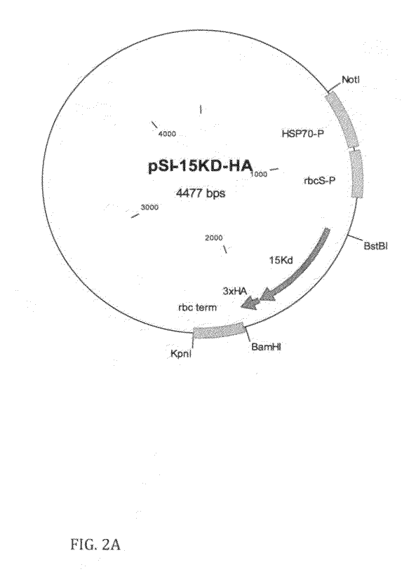

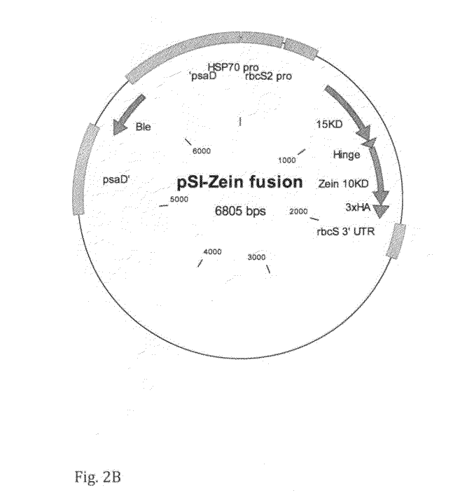

The Zea mays delta zein 15 Kd gene alone (SEQ ID NO: 16) and fused to Zea mays delta zein 10 Kd using the hinge region of anti HSV antibody (accession number: AY191459) as a linker, was synthesized de novo according to Chlamydomonas codon usage (SEQ ID NO: 17). This HSV antibody was previously expressed in Chlamydomonas chloroplasts as was shown by Mayfield et al. (2003). The two cassettes (FIG. 2) were cloned under Chlamydomonas HSP70-rbcS fusion promoter in the plasmid pSI103 (Sizova et al., 2001).

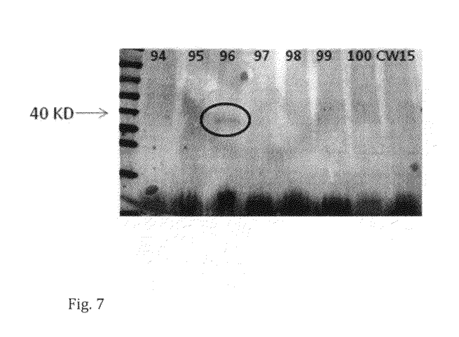

The Zein fusion containing plasmid (FIG. 2B) was transformed to Chlamydomonas CW15 strain and transformants were selected on TAP agar containing 5 μg/ml zeocin. Zeocin resistant colonies were grown in liquid medium and ˜10⁸ cells were taken for further analysis. Expression of the zein protein in these colonies was detected by western analysis using the anti-HA antibody. As shown in FIG. 7, clone #96 expresses a protein of ˜40 kD that is detected by the specific anti-HA antibody. The size of the protein and the fact that it interacts with the specific antibody leads us to conclude that the zein protein is expressed in this colony.

AtCGS and Zein containing plasmids were co-transformed to the arginine-requiring Chlamydomonas strain (CC425) together with p389 plasmid containing the ARG7 gene for complementation.

Colonies transformed with 15 kD-HA and AtCGS-HA that grew on TAP medium without arginine were transferred to new agar plates and screened for transgene existence using PCR with zein and AtCGS specific primers:

(SEQ ID NO: 20)

| | |
|---|---|
| 15KD For: | CGAATTCTTCGAAATGAAGATGGTGATCGTGCTC |

(SEQ ID NO: 21)

| | |
|---|---|
| 15KD REV: | CGGATCCTCACTCGAGGTAGTAGGGCGGGATCGCAG |

(SEQ ID NO: 22)

| | |
|---|---|
| AtCGS For: | CCCGCATCTTCATGGAGAAC |

(SEQ ID NO: 23)

| | |
|---|---|
| AtCGS Rev: | GTGACCGCCGATGTACTTAG |

From around 100 colonies screened by PCR, approximately 53 contained the two cassettes.

PCR positive colonies for both transgenes were selected for western blot analysis using anti HA antibodies (as described in materials and methods part).

In addition to wild type (CC-425) strain the above plasmids are transformed to Chlamydomonas strain containing RNAi or antisense cassette for Rubisco small subunit which reduces Rubisco protein level in the cell. This strain also contains the phytoene desaturase (pds) gene conferring resistance to phytoene desaturase inhibiting herbicides.

For transformation to the marine algae to P. tricornutum, the Zea mays delta zein 15 Kd gene fused to 3xHA is synthetically synthesized according to P. tricornutum codon usage (SEQ ID NO: 19) and cloned downstream to the fcpA promoter in the plasmid pPhaT (Falciatore et al., 1999). The diatom Phaeodactylum tricornutum was transformed by microparticle bombardment using a Biolistic PDS-1000/He Particle Delivery System (Bio-Rad, Hercules, Calif., USA) as previousle described (Falciatore et al., 1999). After about three weeks of incubation under white light, 22-25° C., individual resistant colonies were restraked on 100% ASW+f/2 agar plates, supplemented with 100 μg/ml zeocin (Invitrogen, Carlsbad, Calif., USA) and inoculated into liquid ASW+f/2 medium to be further analyzed.

### Example 2

### Expression of Zea mays Delta Zein 15 kD Protein Alone (SEQ ID NO: 18) or Fused to Zea mays Delta Zein 10 kD Protein (SEQ ID NO: 19) in Chlamydomonas Chloroplasts, Together with Mutated Form of Cystathionine γ-Synthase (CGS) (SEQ ID NO: 15).

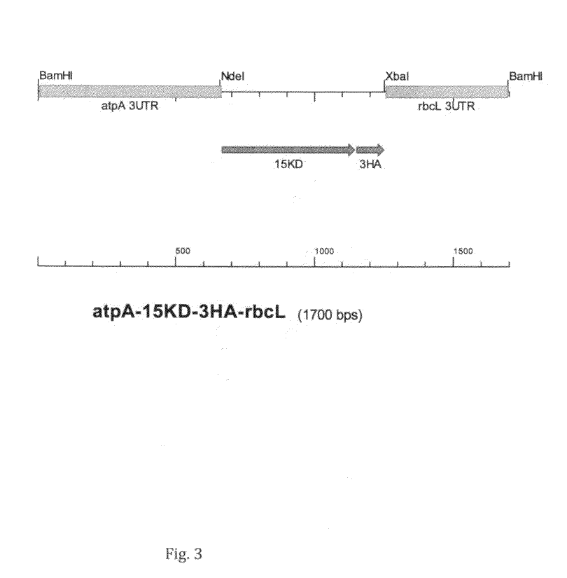

The coding sequence of Zea mays delta zein 151(13-HA alone (SEQ ID NO: 18) or fused to Zea mays delta zein 10 kD using the hinge region of anti HSV antibody (accession number: AY191459) as a linker are de novo synthesized according to Chlamydomonas chloroplast codon usage. This HSV antibody was previously expressed in Chlamydomonas chloroplasts as was shown in Mayfield et al. (2003). The coding sequences are cloned under the control of atpA promoter and rbcL terminator (SEQ ID NO: 10) in plasmid p423 (Chlamydomonas center). The cassettes (FIG. 3) are cloned into the BamHI site in plasmid p322 and transformed into Chlamydomonas chloroplasts together with p228, containing the spectinomycin resistance gene. Chlamydomonas colonies expressing the zein proteins will be screened by western blot with anti HA antibodies and selected transformants are transformed with the mutated form of AtCGS as described in example 1.

### Example 3

### Expression of Corynebacterium dapA Gene (SEQ ID NO: 1) Together with the Gene Encoding BHL8 High Lysine Protein (SEQ ID NO: 3)

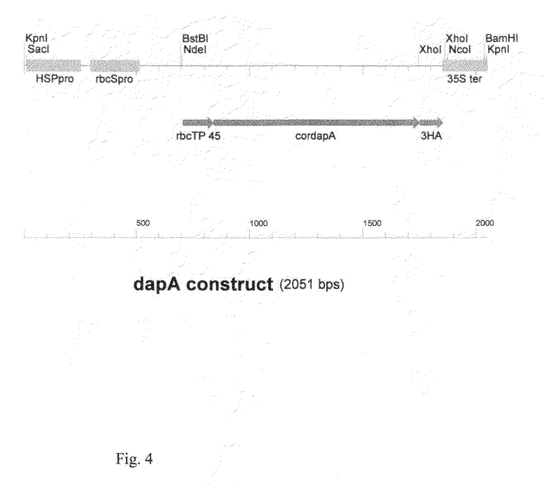

The Corynebacterium gene encoding DHPS (accession number Z21502) fused to the Chlamydomonas rbcS chloroplast transit peptide and 3xHA epitope tag is de novo synthesized according to Chlamydomonas codon usage, and cloned downstream to the Chlamydomonas HSP70-rbcS promoter and upstream to the 35S terminator (FIG. 4). The entire cassette is cloned in pSP124s upstream to the Ble selectable marker.

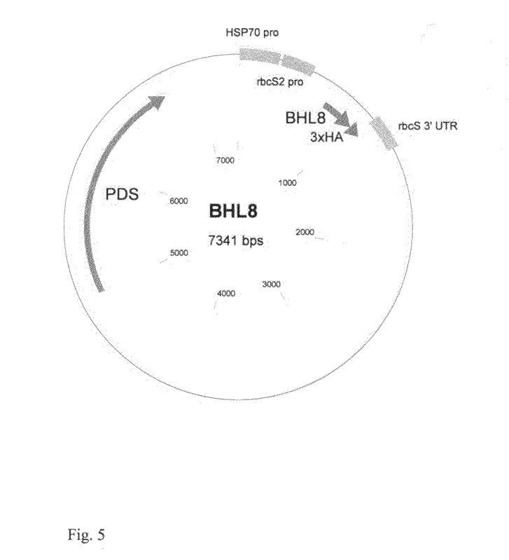

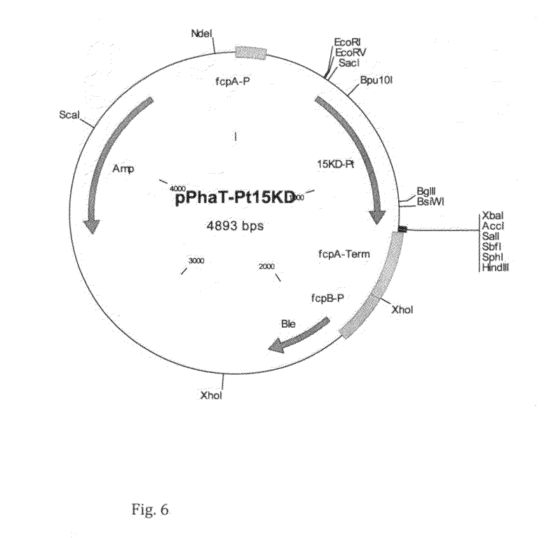

The DNA encoding the BHL8 protein (Jung and Carl, 2000) is de novo synthesized according to Chlamydomonas codon usage and cloned under the Chlamydomonas HSP70-rbcS promoter (Sizova et al., 2001), in a plasmid containing the phytoene desaturase (pds) gene conferring resistance to phytoene desaturase inhibiting herbicides, which may synthesize less beta-carotene (FIG. 5).

Both plasmids are co-transformed to Chlamydomonas and selected on zeocine and flurochloridone containing agar plates.

### Example 4

### Expression of the Anabaena PCC 7120 HetR Gene Linked to High-Lysine α-Helical Coiled-Coil Protein in Synechococcus PCC 7002

The coding sequence of Anabaena HetR is amplified using Anabaena genomic DNA as a template, using the primers: ATGAGTAACGACATCGATCTG (SEQ ID NO: 24) and TTAATCTTCTTTTCTACCAAACAC (SEQ ID NO: 25) and cloned downstream to the Synechococcus rbcL promoter. The cassette comprising the rbcL promoter and HetR CDS is cloned into a PsbA genomic fragment amplified using Synechococcus PCC 7002 genomic DNA as a template. For selection of transformants a kanamycin resistance cassette is cloned downstream to the HetR CDS. The cassette comprising HetR under the control of rbcL promoter, and Kan resistance gene is transformed into Synechococcus PCC 7002 replacing one of at least three redundant endogenous PsbA genes. Transformants resistant to kanamycin are selected for amino acid analysis.

### Example 5

### Expression of Anabaena PCC 7120 HetR Gene Linked to Zea mays Delta Zein 15 kD Protein Fused to Zea mays Delta Zein 10 kD Protein Together with Mutated Form of Arabidopsis Cystathionine γ-Synthase (CGS)

The D-AtCGS coding sequence (SEQ ID NO: 6) (Hacham et al., 2006), fused to Chlamydomonas rbcS chloroplast transit peptide, is chemically synthesized according to Chlamydomonas codon usage and cloned downstream to the Chlamydomonas HSP70-rbcS promoter and upstream to 35S terminator. The entire cassette is cloned into pSP124s upstream to the Ble selectable marker (FIG. 1).

The Anabaena HetR CDS (SEQ ID NO: 7) linked to the Zein fusion cassette described in Example 1 is de novo synthesized according to Chlamydomonas codon usage and is cloned downstream to the Chlamydomonas HSP70-rbcS promoter (SEQ ID NO:12) (Sizova et al., 2001), in plasmid containing the phytoene desaturase (pds) gene conferring resistance to phytoene desaturase inhibiting herbicides.

Both plasmids are co-transformed to Chlamydomonas and selected on zeocine and flurochloridone containing agar plates.

### REFERENCES

Avraham, T., Badani, H., Galili, S., and Amir, R. (2005). Enhanced levels of methionine and cysteine in transgenic alfalfa (Medicago sativa L.) plants over-expressing the Arabidopsis cystathionine gamma-synthase gene. Plant Biotechnol J 3, 71-79.
Blake, M. S., Johnston, K. H., Russell-Jones, G. J. and Gotschlich, E. C. (1984). A rapid, sensitive method for detection of alkaline phosphatase-conjugated anti-antibody on Western blots, Anal Biochem 136, 175-9.
Chakraborty, S., Chakraborty, N., and Datta, A. (2000). Increased nutritive value of transgenic potato by expressing a nonallergenic seed albumin gene from Amaranthus hypochondriacus. Proceedings of the National Academy of Sciences USA 97, 3724-3729.
Chui, C.-F. C., Falco, S. C., Rice, J. A. and Knowlton, S. (2003) High sulphur seed protein gene and method for increasing the sulphur amino acid content of plants. Canadian Patent CA 2104022.
Falciatore, A., Casotti, R., Leblanc. C., Abrescia, C., Bowler, C. (1999) Transformation of nonselectable reporter genes in marine diatoms. Mar. Biotechnol. 1:239-251
Fischer, H., Robl, I. Sumper, M, and Kroeger, N. (1999). Targeting and covalent modification of cell wall and membrane proteins heterologously expressed in the diatom Cylindrotheca fusiformis (Bacillariophyceae), Journal of Phycology 35, 115-120.
Grzebyk, D., O. Schofield, P. Falkowski, and J. Bernhard (2003) The Mesozoic radiation of eukaryotic algae: the portable plastid hypothesis. J. Phycol. 39:259-267.
Hacham, Y., Schuster, G., and Amir, R. (2006). An in vivo internal deletion in the N-terminus region of Arabidopsis cystathionine gamma-synthase results in CGS expression that is insensitive to methionine. Plant J 45, 955-967.
Hacham, Y., Matityahu, I., Schuster, G., and Amir, R. (2008). Overexpression of mutated forms of aspartate kinase and cystathionine gamma-synthase in tobacco leaves resulted in the high accumulation of methionine and threonine. Plant J 54, 260-271.
Jung, R., and Falco, S. C. (2000). Transgenic corn with an improved amino acid composition. 8th International Symposium on Plant seeds. Gatersleben, Germany.
Karchi H, Shaul O, and Galili G, (1994) Lysine synthesis and catabolism are coordinately regulated during tobacco seed development. Proceedings of the National Academy of Sciences USA 91: 2577-2581
Keeler, S., Maloney, C., Webber, P., Patterson, C., Hirata, L., Falco, S., and Rice, J. (1997). Expression of de novo high-lysine a-helical coiled-coil proteins may significantly increase the accumulated levels of lysine in mature seeds of transgenic tobacco plants. Plant Molecular Biology 34, 15-29.
Kindle, K. L. (1990). High-frequency nuclear transformation of Chlamydomonas reinhardtii. Proceedings of the National Academy of Sciences USA 87, 1228.
Kirihara, J. A., Hibberd, K. A. and Janice, A. (2001) Method for altering the nutritional content of plant seed. U.S. Pat. No. 6,326,527.
Lumbreras, V., Stevens, D. R., and Purton, S. (1998). Efficient foreign gene expression in Chlamydomonas reinhardtii mediated by an endogenous intron. Plant Journal 14, 441-447.
Masumura, T., Shibata, D., Hibino, T., Kato, T., Kawabe, K., Takeba, G., Tanaka, K., and Fujii, S. (1989). cDNA cloning of an messenger-RNA encoding a sulfur-rich 10 kDa prolamin polypeptide in rice seeds. Plant Molecular Biology 12, 123-130.
Mayfield, S., Franklin, S., and Lerner, R. (2003) Expression and assembly of a fully active antibody in algae. Proceedings of the National Academy of Sciences USA 100, 438-42
Morris, C. E. and Sands, D. C. (2006) The breeders' dilemma—yield or nutrition Nature Biotechnology 24, 1078-1080.
Roesler K R, Rao A G (2000) A single disulfide bond restores thermodynamic and proteolytic stability to an extensively mutated protein. Protein Sci 9: 1642-1650
Roesler, K., and Rao, A. (2001). Rapid gastric fluid digestion and biochemical characterization of engineered proteins enriched in essential amino acids. Journal of Agricultural and Food Chemistry 49, 3443-3451.
Sizova, I., Fuhrmann, M., and Hegemann, P. (2001). A Streptomyces rimosus aphVIII gene coding for a new type phosphotransferase provides stable antibiotic resistance to Chlamydomonas reinhardtii. Gene 277, 221-229.
Zhu, X., and Galili, G. (2004). Lysine metabolism is concurrently regulated by synthesis and catabolism in both reproductive and vegetative tissues. Plant Physiol 135, 129-136.

What is claimed is:

1. A method to produce improved animal feed, said method comprising the steps of:
a. Genetically modifying alga or cyanobacterium methionine and/or lysine biosynthesis to upregulate production of methionine and/or lysine;
b. Transforming the alga or cyanobacterium with a nucleotide sequence encoding a high methionine and/or lysine protein for a sink of the upregulated methionine and/or lysine;
c. Cultivating the alga or cyanobacterium in an axenic culture;
d. Harvesting cultured algae or cyanobacteria; and
e. Providing the algae or cyanobacteria for animal feed.

2. The method of claim 1, wherein upregulation of lysine is achieved by transforming the alga or cyanobacterium with RNAi of algal lysine-ketoglutarate reductase/saccharopine dehydrogenase.

3. The method of claim 1, wherein upregulation of methionine is achieved by transforming the alga or cyanobacteria with mutated Arabidobsis cystathionine γ-synthase.

4. The method of claim 1, wherein the protein is a high lysine protein and is selected from the group consisting of BHL8 protein, AmA1 seed protein, and coiled-coil high lysine/high methinone protein.

5. The method of claim 4, wherein gene encoding the protein is expressed in nuclear or in chloroplast genome of the algae or cyanobacteria.

6. The method of claim 1, wherein the protein is a high methionine protein and is selected from the group consisting of 2S albumin protein, het R encoding protein, coiled-coil high lysine/high methinone protein, or delta zein structural 15/10/18 protein

7. The method of claim 6, wherein gene encoding the protein is expressed in nuclear or chloroplast genome of the algae or cyanobacteria.

8. The method of claim 1, wherein the alga is selected from the group consisting of Phaeodactylum tricornutum, Amphiprora hyaline, Amphora spp., Chaetoceros muelleri, Navicula saprophila, Nitzschia sp., Nitzschia communis, Scenedesmus dimorphus, Scenedesmus obliquus, Tetraselmis suecica, Chlamydomonas reinhardtii, Chlorella vulgaris, Haematococcus pluvialis, Neochloris oleoabundans, Botryococcus braunii, Botryococcus sudeticus, Nannochloropsis oculata, Nannochloropsis salina, Nannochloropsis spp., Nannochloropsis gaditana, Nannochloris spp., Isochrysis aff galbana, Euglena gracilis, Neochloris oleoabundans, Nitzschia palea, Pleurochrysis carterae, and Tetraselmis chuii.

9. The method of claim 1, wherein the cyanobacterium is selected from the group consisting of Aphanocapsa sp., Gloeobacter violaceus PCC7421, Synechococcus elongatus PCC6301, Synechococcus. PCC7002, Synechococcus. PCC7942, and Synechosystis PCC6803, Thermosynechococcus elongatus BP-1, Spirulina sp.

10. A transgenic algae or cyanobacterium having an upregulated biosynthesis of methionine and/or lysine.

11. The transgenic alga or cyanobacterium of claim 10, wherein the alga or cyanobacterium has an upregulated biosynthesis of lysine and the upregulation is achieved by transforming the alga or cyanobacterium with RNAi of algal lysine-ketoglutarate reductase/saccharopine dehydrogenase.

12. The transgenic alga or cyanobacterium of claim 10, wherein the alga or cyanobacterium has an upregulated biosynthesis of methionine and the upregulation is achieved by transforming the alga or cyanobacteria with mutated Arabidobsis cystathionine γ-synthase.

13. The transgenic alga or cyanobacterium of claim 10, wherein the cyanobacterium or alga additionally expresses a recombinant protein naturally rich with methionine and lysine as a sink for upregulated methionine and/or lysine biosynthesis.

14. The transgenic alga or cyanobacterium of claim 10, wherein said alga or cyanobacterium is transformed with a polynucleotide sequence encoding a protein selected from the group consisting of BHL8 protein, AmA1 seed protein, coiled-coil high lysine/high methinone protein, 2S albumin protein, Zea mays delta zein proteins and hetR gene encoding protein.

15. The transgenic alga or cyanobacterium of claim 14, wherein gene encoding the protein is expressed in nuclear or chloroplast genome of the alga or cyanobactrium.

16. The transgenic alga or cyanobactrium of claim 14, wherein the alga or cyanobactrium is further modified to express reduced level of Rubisco protein.

17. The transgenic alga or cyanobacterium of claim 16, wherein the reduced level Rubisco protein is achieved by transforming the cells with a vector comprising rbcS encoding polynucleotides in an antisense or in an RNAi-construct under a constitutive promoter.

18. Animal feed composition comprising transgenic algae or cyanobacteria having a modified biosynthesis of methionine and/or lysine and expressing recombinant protein with high lysine and/or methionine content.

19. Animal feed composition comprising recombinant protein produced in algae or cyanobacteria.

20. The animal feed composition of claim 18 or, wherein the feed is used for mammals.

21. The animal feed composition of claim 18, wherein the feed is used for fowl.

22. The animal feed composition of claim 18, wherein the feed is used for fish.

23. The animal feed of claim 18, wherein the feed is used for carnivorous fish.


SEQUENCE LISTING (24 sequences)

SEQ ID NO: 1 - DNA, 909 bp, Corynebacterium
  CDS (1)..(909): coding region of feedback insensitive bacterial DHDPS
atgagcacaggtttaacagctaagaccggagtagagcacttcggcaccgttggagtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgagtctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgctctgcgtctgcagggcatcaacgtaggagatcctcgacttccaattatggctccaaatgagcaggaacttgaggctctccgagaagacatgaaaaaagctggagttctactcgag

SEQ ID NO: 2 - protein, 303 aa, Corynebacterium
MSTGLTAKTGVEHFGTVGVAMVTPFTESGDIDIAAGREVAAYLVDKGLDSLVLAGTTGESPTTTAAEKLELLKAVREEVGDRAKLIAGVGTNNTRTSVELAEAAASAGADGLLVVTPYYSKPSQEGLLAHFGAIAAATEVPICLYDIPGRSGIPIESDTMRRLSELPTILAVKDAKGDLVAATSLIKETGLAWYSGDDPLNLVWLALGGSGFISVIGHAAPTALRELYTSFEEGDLVRAREINAKLSPLVAAQGRLGGVSLAKAALRLQGINVGDPRLPIMAPNEQELEALREDMKKAGVLLE

SEQ ID NO: 3 - protein, 448 aa, Chlamydomonas reinhardtii
  PEPTIDE (1)..(448): lysine-ketoglutarate reductase/saccharopine dehydrogenase
MRRVANTSRATGARCQGAKLVARPCARRAAVHVICATGPVPDKSVVVIGGTGRVGSSTAATLLKEFPNLKVTVASRSDDSFKAAVERRPELSKAGFQRVDITNADSVQALLKSTGADLVIHTAGPFQRSKNYAVLEAAIASGTGYIDVCDDTPFAEGAKAAYMEKAKAAGVPAIVSGGIYPGTSNVMAAHIISIARAEYDDNWNYRTPAPGESVEPKWLRYSYYTAGSGGAGPTILETSFLLAGEDVIVYKDNKEVVLPPISNRREVDFGPGVGRKGVYLYNLPEVVSGHKYMRVPDVSARFGTDPFIWNWAMWLTARLVPRSLLNDRNFVKGFAKLSDPFVRNVDKIIGEAVAMRVEVDMVGGKNSSGIFVHKYLSQSMGYSTAAFAQSVLQGKTQPGVWYPEEKEALQDRRQFLQFAATGCSRFELNRSAWALESEIKQIGGMIYW

SEQ ID NO: 4 - protein, 67 aa, Chlamydomonas reinhardtii
  PEPTIDE (1)..(67): BHL8
MAKMKCTWPELVGKTVEKAKKMIMKDKPEAKIMVLPVGTKVTGEWKMDRVRLWVDKKDKIAKTPKCG

SEQ ID NO: 5 - protein, 147 aa, artificial
  chemically synthetized
ATGGAGGAGAAGCTGAAGGCCATGGAGGAGAAGCTGAAGGCCATGGAGGAGAAGCTGAAGGCCATGGAGGAGAAGCTGAAGGCCATGGAGGAGAAGCTGAAGGCCATGGAGGAGAAGCTGAAGGCCATGGAGGAGAAGATGAAGGCC

SEQ ID NO: 6 - protein, 304 aa, Amaranthus hypochondriacus
  PEPTIDE (1)..(304): seed protein AmA1
MAGLPVIMCLKSNNNQEYLRYQSDNIQQYGLLQFSADKILDPLAQFEVEPSKTYDGLVHIKSRYTNKYLVRWSPNHYWITASANEPDENKSNWACTLFKPLYVEEGNMKKVRLLHVQLGHYTENYTVGGSFVSYLFAESSQIDTGSKDVFHVIDWKSIFQFPKTYVTFKGNNGKYLGVITINQLPCLQFGYDNLNDPKVAHQMFVTSNGTICIKSNYMNKFWRLSTDNWILVDGNDPRETNEAAALFRSDVHDFNVISLLNMQKTWFIKRFTSGKPEFINCMNAATQIVDETAILEIIELGSNN

SEQ ID NO: 7 - protein, 533 aa, Arabidopsis thaliana
  PEPTIDE (1)..(533): D-AtCGS Mutated form of Arabidopsis cystathionine c-synthase
MAVSSFQCPTIFSSSSISGFQCRSDPDLVGSPVGGSSRRRVHASAGISSSFTGDAGLSSRILRFPPNFVRQLSIKARRNCSNIGVAQIVAAKWSNNPSSAAPVAAAPPVVLKSVDEEVVVAEEGIREKIGSVQLTDSKHSFLSSDGSLTVHAGERLGRGIVTDAITTPVVNTSAYFFKKTAELIDFKEKRSVSFEYGRYGNPTTVVLEDKISALEGAESTLVMASGMCASTVMLLALVPAGGHIVTTTDCYRKTRIFMENFLPKLGITVTVIDPADIAGLEAAVNEFKVSLFFTESPTNPFLRCVDIELVSKICHKRGTLVCIDGTFATPLNQKALALGADLVVHSATKYIGGHNDVLAGCICGSLKLVSEIRNLHHVLGGTLNPNAAYLIIRGMKTLHLRVQQQNSTAFRMAEILEAHPKVSHVYYPGLPSHPEHELAKRQMTGFGGVVSFEIDGDIETTIKFVDSLKIPYIAPSFGGCESIVDQPAIMSYWDLPQEERLKYGIKDNLVRFSFGVEDFEDVKADILQALEAI

SEQ ID NO: 8 - DNA, 914 bp, Anabeana
  misc_feature (1)..(914): HetR gene from Anabaena sp. modified according to Chlamydomonas reinhardtii codon usage
atggccggcctgcccgtgatcatgtgcctgaagagcaacaacaaccaggagtacctgcgctaccagagcgacaacatccagcagtacggcctgctgcagttcagcgccgacaagatcctggaccccctggcccagttcgaggtggagcccagcaagacctacgacggcctggtgcacatcaagagccgctacaccaacaagtacctggtgcgctggagccccaaccactactggatcaccgccagcgccaacgagcccgacgagaacaagagcaactgggcctgcaccctgttcaagcccctgtacgtggaggagggcaacatgaagaaggtgcgcctgctgcacgtgcagctgggccactacaccgagaactacaccgtgggcggcagcttcgtgagctacctgttcgccgagagcagccagatcgacaccggcagcaaggacgtgttccacgtgatcgactggaagagcatcttccagttccccaagacctacgtgaccttcaagggcaacaacggcaagacctgggcgtgatcaccatcaaccagctgccctgcctgcagttcggctacgacaacctgaacgaccccaaggtggcccaccagatgttcgtgaccagcaacggcaccatctgcatcaagagcaactacatgaacaagttctggcgcctgagcaccgacaactggatcctggtggacggcaacgacccccgcgagaccaacgaggccgccgccctgttccgcagcgacgtgcacgacttcaacgtgatcagcctgctgaacatgcagaagacctggttcatcaagcgcttcaccagcggcaagcccgagttcatcaactgcatgaacgccgccacccagatcgtggacgagaccgccatcctggagatcatcgagctgggcagcaacaactaa

SEQ ID NO: 9 - protein, 178 aa, Zea mays
  PEPTIDE (1)..(178): Zea mays delta zein structural 15 protein
MKMVIVLVVCLALSAASASAMQMPCPCAGLQGLYGAGAGLTTMMGAGGLYPYAEYLRQPQCSPLAAAPYYAGCGQPSAMFQPLRQQCCQQQMRMMDVQSVAQQLQMMMQLERAAAASSSLYEPALMQQQQQLLAAQGLNPMAMMMAQNMPAMGGLYQYQLPSYRTNPCGVSAAIPPYY

SEQ ID NO: 10 - DNA, 2603 bp, artificial
  chemically synthetized
ggatccgactttattagaggcagtgtttatatacctaaacgtcaaaagtcatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttatggcaatgcgtactccagaagaacttagtaatcttattaaagatttaattgaacaatacactccagaagtgaaacatatgcgtcagctgagcattaaagcccgtagaaactgtagcaacatcggtgttgcacagatcgtggcggctaagtggtccaacaacccatcctccgccgcccctgtggctgccgcgcctcccgtcgtgctgaaaagcgtcgatgaggaggttgtggtggccgaggaggggatcagggagaagataggtagtgtacagctgacggattccaaacattctttcttgagctccgatgggagcctcactgttcatgccggtgaaagattaggccgtggtatagtgacggatgctatcactactcctgtagtcaacacatctgcctacttcttcaagaaaactgctgagcttattgacttcaaggagaaaaggagtgtgagtttcgagtatggtcgttatggtaaccctacgactgtggtacttgaagataagataagtgcacttgaaggggctgaatcaactttggttatggcatctgggatgtgtgcaagcactgttatgcttttggcattggttcctgctggtggacacattgtcactactactgattgctacaggaagactaggatcttcatggagaattttcttcccaagttggggatcactgtcactgtgattgatcctgctgatatcgcagggcttgaagctgcagtgaatgagttcaaggtatctctgttcttcactgagtccccgacaaacccattccttagatgtgtcgacattgagctagtttcaaaaatatgccacaagaggggaactctggtttgcattgatggcacctttgcaacacctctgaatcagaaagcccttgctcttggtgctgatcttgtcgtgcactctgctacaaagtacattggaggacacaatgatgttcttgctggatgcatctgtggttcactgaagttggtttcagaaattcgcaatctgcatcatgtgttgggaggaacacttaacccaaacgctgcgtacctaatcatccgaggcatgaagacattgcatcttcgtgtacagcaacagaattcgaccgcttttagaatggccgaaattttagaggcacatcctaaggtgagtcatgtgtactatccaggccttccaagtcatcccgaacatgaactcgccaagcgacaaatgactggttttggaggtgtggtcagtttcgagattgatggagacattgaaacgacaatcaagtttgtggattctctaaagattccttacattgcaccatccttcggtggctgcgaaagcattgttgaccaacctgctatcatgtcctactgggatctgccgcaagaggagagactaaagtatggaatcaaagataacttggttcgtttcagctttggagttgaagactttgaagatgtcaaagctgacattcttcaagctctcgaagccatctacccatacgatgttcctgactatgcgggctatccctatgacgtcccggactatgcaggatcctatccatatgacgttccagattacgctgctcagtagtctagatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatactaaagcggatgtaactcaatcggtagagtgcgatccttccaagttcgaggttgtgggttcgagtcccatcatccgctaaaccaatctataaaagttgttgaatatgctgaaatgttttcaaagaaaaagcctagtttttcttttacaacaagcaaagaacaattggcattctttgattgtaagaaaatgcgcttggatcc

SEQ ID NO: 11 - DNA, 1238 bp, artificial
  chemically synthetized
aagcttttcgaaatgtccaacgatattgatctgattaagcggctgggtccgtcggccatggaccagatcatgctgtacctggccttcagcgcgatgcggacgagcggccaccgccacggcgccttcctcgacgctgccgcgacggctgcgaagtgcgcgatctacatgacctacctcgagcagggccagaacctccgcatgaccggccacctccaccacctcgagcccaagcgcgtcaagatcattgtcgaggaggtccggcaagccctgatggagggcaagctgctcaagaccctggggagccaggagccccgctacctgatccagttcccctacgtgtggatggagcagtacccgtggattcccgggcggtcccgcatccccggcaccagcctgacgtcggaggagaagcgccagatcgagcacaagctgccgagcaacctgccggacgcgcagctggtgacctcgtttgagtttctggagctgatcgagtttctgcacaagcggagccaggaggacctgcctccggagcatcgcatggagctgtcggaggcgctggcggagcacatcaagcgccgcctgctgtactccggcacggtgacccgcatcgactccccctggggtatgcccttctatgcgctgactcgccccttctacgctcccgccgacgatcaggagcggacctacatcatggtggaggacactgcccgctacttccgcatgatgaaggactgggccgagaagcgccccaacgcgatgcgcgctctggaggagctcgacgtgcctcccgagcgctgggacgaggccatgcaagagctggacgagatcatccgcacgtgggccgacaagtaccaccaggtgggcggcatcccgatgattctgcagatggtgttcggccgcaaggaggacgtgccgtcgacgccgcctaccccgtccccgagcacccctcccaccccctccccctcgatggaggagaagctgaaggccatggaggagaagctgaaggctatggaggagaagctgaaggccatggaggagaagctgaaggcgatggaggagaagctgaaggccatggaggagaagctgaaggccatggaggagaagatgaaggcgctggagtatccctacgacgtgcccgactacgcgggctacccctacgacgtgcccgactacgccggcagctacccgtacgacgtgccggactacgccgctcagtaactcgagtgagatccggatcc

SEQ ID NO: 12 - protein, 135 aa, Chlamydomonas
  PEPTIDE (1)..(135): Chlamydomonas rbS transit peptide
ATGGCCGCCGTCATTGCCAAGTCCTCCGTCTCCGCGGCCGTGGCCCGCCCGGCCCGCTCCAGCGTGCGCCCCATGGCCGCGCTGAAGCCCGCCGTCAAGGCCGCCCCCGTGGCTGCCCCGGCTCAGGCCAACCAG

SEQ ID NO: 13 - protein, 99 aa, artificial
  chemically synthetized
TACCCGTACGATGTCCCGGACTACGCCGGCTATCCCTACGATGTGCCTGACTACGCGGGCTCCTACCCCTACGACGTGCCCGACTACGCTGCCCAGTAG

SEQ ID NO: 14 - DNA, 739 bp, Zea mays
  misc_feature (1)..(739)
  misc_feature (1)..(739): gene encoding Zea mays delta zein structural 15 protein
tccagatcagcaaagcggcagtgcgtagagaggatcgtcgaacagaacagcatgaagatggtcatcgttctcgtcgtgtgcctggctctgtcagctgccagcgcctctgcaatgcagatgccctgcccctgcgcggggctgcagggcttgtacggcgctggcgccggcctgacgacgatgatgggcgccggcgggctgtacccctacgcggagtacctgaggcagccgcagtgcagcccgctggcggcggcgccctactacgccgggtgtgggcagccgagcgccatgttccagccgctccggcaacagtgctgccagcagcagatgaggatgatggacgtgcagtccgtcgcgcagcagctgcagatgatgatgcagcttgagcgtgccgctgccgccagcagcagcctgtacgagccagctctgatgcagcagcagcagcagctgctggcagcccagggtctcaaccccatggccatgatgatggcgcagaacatgccggccatgggtggactctaccagtaccagctgcccagctaccgcaccaacccctgtggcgtctccgctgccattccgccctactactgattcatgatatttgggaaatctcctctatccatctctctctatctatatatgtaataatgcagtaagacgacacacattatcatgtgtggtatgaccaataatatatgcatggtcataataaagttttggttttaaaaaaaaaaaaaaaaaa

SEQ ID NO: 15 - DNA, 1146 bp, artificial
  chemically synthetized
atgaagatggtgatcgtgctcgtcgtgtgcctcgccctgtccgcggctagcgcgagcgccatgcagatgccgtgcccctgcgctggcctgcagggcctgtacggtgcgggtgccggcctgacgacgatgatgggggctggcgggctgtacccgtacgccgagtacctccggcagccccagtgctcgccgctggccgctgccccctactatgcgggctgcggccagcccagcgccatgttccaacccctgcgccagcagtgctgccagcagcagatgcgcatgatggacgtccagagcgtggcgcagcagctgcaaatgatgatgcagctggagcgcgctgctgccgcctccagctccctgtacgagcccgctctgatgcagcagcagcaacagctgctggctgcccagggcctgaaccccatggcgatgatgatggcccagaacatgcccgcgatgggtggcctgtaccagtaccagctgccctcgtaccgcaccaacccgtgcggcgtgtcggctgcgatcccgccctactacgtgccctcgacgccccctaccccctccccctcgacccctccgaccccttccccatcgatggcggccaagatgctggcgctgtttgcgctgctggccctgtgcgcctcggccaccagcgccactcacattcccggccacctgcctccggtgatgcccctgggcacgatgaacccgtgcatgcaatactgcatgatgcaacagggcctggccagcctcatggcgtgcccgtccctgatgctccaacagctcctggccctgcccctgcagaccatgccggtgatgatgccccagatgatgaccccgaacatgatgtcgccgctgatgatgcctagcatgatgagccccatggtcctgccctccatgatgagccagatgatgatgccgcagtgccactgcgacgcggtgagccagatcatgctgcagcagcagctccccttcatgttcaacccgatggccatgaccatcccgcctatgtttctgcagcagcccttcgtgggcgccgcctttctcgagtacccgtacgacgtccctgactacgcgggctacccctacgatgtgcccgactacgcgggctcctatccctacgacgtgccggactacgctgcgcagtga

SEQ ID NO: 16 - DNA, 534 bp, Zea mays
  CDS (1)..(534): 15kD Zein according to Chlamydomonas chloroplast codon usage
atgaaaatggtaattgttcttgtagtttgtttagcattaagtgctgcatcagctagtgcaatgcaaatgccttgtccatgtgctggtttacaaggtttatatggtgctggtgctggattaactacaatgatgggagctggtggtttatatccttatgctgaatatttacgtcaaccacaatgttctccattagctgcagctccatattacgcaggttgtggtcaaccaagtgctatgtttcaaccattacgtcaacaatgttgtcaacaacaaatgcgtatgatggatgttcaatctgtagctcaacaattacaaatgatgatgcaattagaaagagcagctgcagcttcaagttcattatatgaacctgcattaatgcaacaacaacaacaattacttgctgcacaaggtttaaatcctatggctatgatgatggctcaaaatatgcctgctatgggtggtttatatcaataccaattaccaagttatcgtacaaacccatgtggtgtatctgctgctattccaccatattac

SEQ ID NO: 17 - protein, 178 aa, Zea mays; identical to SEQ ID NO: 9

SEQ ID NO: 18 - DNA, 813 bp, artificial
  chemically synthetized
atgagatccttttgcatcgcagcccttttggctgtggcatctgccttcaccacacagccaacttccttcactgtgaagactgcgaatgtgggcgaacgggcgagtggggttttccctgagcagtcttctgctcatcgcacgcgtaaagcaacgattgtcatgaagatggtcatcgtcctcgtcgtctgcctcgccttgtccgccgcctccgcctccgccatgcagatgccctgtccctgcgccggactccagggactctacggtgccggtgccggactcaccaccatgatgggagccggtggactctacccctacgccgaatacctccgtcagccccagtgctcccctttggccgccgccccctactacgccggatgtggacagccgtccgccatgttccagcccttgcgtcagcagtgctgccagcagcagatgcgtatgatggacgtccagtccgtcgcccagcagctccagatgatgatgcagctcgaacgtgccgccgccgcctcctcctccttgtacgaacccgccctcatgcagcagcagcaacagttgctcgccgcccagggactcaaccccatggccatgatgatggcccagaacatgcccgccatgggaggactctaccagtaccagctcccctcctaccgtaccaacccctgtggtgtctccgccgccattcccccgtactacagatctggtggaggtggcggatacccgtacgacgtccctgattacgccggatacccttacgatgtcccggactacgccggttcctacccctacgacgtcccggactacgccgcccagtaa

SEQ ID NO: 19 - DNA, 34 bp, artificial
  chemically synthetized
cgaattcttcgaaatgaagatggtgatcgtgctc

SEQ ID NO: 20 - DNA, 36 bp, artificial
  chemically synthetized
cggatcctcactcgaggtagtagggcgggatcgcag

SEQ ID NO: 21 - DNA, 20 bp, artificial
  chemically synthetized
cccgcatcttcatggagaac

SEQ ID NO: 22 - DNA, 20 bp, artificial
  chemically synthetized
gtgaccgccgatgtacttag

SEQ ID NO: 23 - DNA, 21 bp, artificial
  chemically synthetized
atgagtaacgacatcgatctg

SEQ ID NO: 24 - DNA, 24 bp, artificial
  chemically synthetized
ttaatcttcttttctaccaaacac